(19) United States
(12) Patent Application Publication
(10) Pub. No.: US 2026/0130898 A1
(43) Pub. Date: May 14, 2026

(54) AMINONAPHTHOQUINONE COMPOUNDS FOR TREATMENT AND/OR PREVENTION OF OPTIC NEUROPATHY

(71) Applicant: Yun YEN, Arcadia, CA (US)

(72) Inventor: Yun YEN, Arcadia, CA (US)

(21) Appl. No.: 18/947,302

(22) Filed: Nov. 14, 2024

(51) Int. Cl. A61K 31/4409; A61P 27/02

(52) U.S. Cl. CPC A61K 31/4409; A61P 27/02

(57) ABSTRACT

The present disclosure provides methods and uses of aminonaphthoquinone compounds in treatment and/or prevention of an optic neuropathy.

### FIELD OF THE INVENTION

The present disclosure relates to a field of disease. Particularly, the present disclosure provides methods and uses of aminonaphthoquinone compounds in treatment and/or prevention of an optic neuropathy.

### BACKROUND OF THE INVENTION

Retinal ganglion cells (RGCs) are essential components of the visual system that transmit visual information from the retina to the brain. Maintaining the health of RGCs and optimizing their physiological function is crucial for normal vision. However, various ocular diseases can cause RGC death, resulting in visual impairment or loss. Ischemic optic neuropathy is characterized as a reduction of obstruction in blood flow to the optic nerve, which results in RGC apoptosis and visual morbidity. The pathophysiological mechanisms contributing to RGC death are complex, involving numerous interconnected pathways such as excitotoxicity, oxidative stress, and inflammatory response. Understanding the complicated mechanism of RGC death in ischemia injury is vital for developing therapeutic strategies to preserve RGC function and improve visual recovery. Targeting these pathways has the potential to prevent or attenuate RGC damage while also minimizing the impact of ischemia injury on visual function.

### SUMMARY OF THE INVENTION

The present disclosure is, at least in part, based on the discovery of using aminonaphthoquinone compounds and their effective dose in the prevention and/or treatment of an optic neuropathy associated with apoptotic death of retinal ganglion cells (RGCs).

In one embodiment, the present disclosure provides a method for preventing and/or treating an optic neuropathy associated with apoptotic death of RGCs, comprising administering an effective amount of a compound of Formula (I) described herein or a pharmaceutically acceptable salt, hydrate or isomer as an active ingredient to the subject,

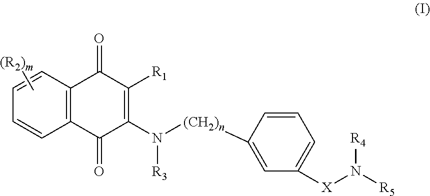

wherein
R₁ is halogen;
each R₂ is the same or different, representing H, C₁₋₁₀alkyl, C₂₋₁₀alkenyl, C₂₋₁₀alkynyl, NH₂, NO₂, C₁₋₁₀alkyloxy, C₁₋₁₀alkylthio, C₁₋₁₀alkylamino, C₁₋₁₀alkyloxyC₁₋₁₀alkyl, OH or CN, C₆₋₁₀aryl or C₅₋₇heterocyclic having 1 to 3 heteroatoms selected from the group consisting of N, O and S;
R₃ is H, C₁₋₁₀alkyl, C₂₋₁₀alkenyl, C₂₋₁₀alkynyl, NH₂, NO₂, OH or CN;
R₄ is H, C₁₋₁₀alkyl, C₂₋₁₀alkenyl, C₂₋₁₀alkynyl, NH₂, NO₂, OH or CN;
R₅ is OH, C₃₋₈cycloalkyl, phenyl unsubstituted or substituted with one to three same or different substituents selected from OH, CN, halogen, NH₂ or C₁₋₄alkylpiperazinyl, C₁₋₆alkylpiperazinyl, C₁₋₆alkylpyridinyl, C₁₋₆alkylpyrrolidinyl, pyridinyl, pyrimidinyl, pyrazinyl. piperazinyl, pyrrolidinyl, thiazolyl, benzimidazolyl, pyrazolyl, indazolyl, quinolinyl, indolyl, azaindolyl, azaindazolyl, deazapurinyl, indanyl, morpholinoyl or C₁₋₄alkylmorpholinoyl, each of which is unsubstituted or substituted with one, two or three groups selected from OH, CN, halogen, NO₂, C₁₋₄alkyl, or NH₂;
X is —C(O)
Y is —N—;
m is an integer of 0-3; and
n is an integer of 1-7.

The present disclosure also provides a pharmaceutical composition for use in a method for preventing and/or treating an optic neuropathy associated with apoptotic death of RGCs, wherein the pharmaceutical composition comprises a compound of Formula (I) described herein. Also provided is use of a pharmaceutical composition in the manufacture of a medicament for preventing and/or treating an optic neuropathy associated with apoptotic death of RGCs, wherein the pharmaceutical composition comprises a compound of Formula (I) described herein.

In some embodiments, m is 0; Ri is halogen; n is any integer of 1-4; R₃ is H; X is C(O); R₄ is H; and R₅ is OH, C₃₋₈cycloalkyl, phenyl unsubstituted or substituted with one to three same or different substituents selected from OH, CN, halogen, NH₂ or C₁₋₄alkylpiperazinyl, C₁₋₆alkylpiperazinyl, C₁₋₆alkylpyridinyl, C₁₋₆alkylpyrrolidinyl, pyridinyl, pyrimidinyl, pyrazinyl. piperazinyl, pyrrolidinyl, thiazolyl, benzimidazolyl, pyrazolyl, indazolyl, quinolinyl, indolyl, azaindolyl, azaindazolyl, deazapurinyl, indanyl, morpholinoyl or C₁₋₄alkylmorpholinoyl, each of which is unsubstituted or substituted with one, two or three groups selected from OH, CN, halogen or NH₂.

In some embodiments, m is 0; R₁ is halogen; n is any integer of 1-2; R₃ is H; X is C(O); R₄ is H; and R₅ is OH, C₃₋₈cycloalkyl, pyridinyl, phenyl substituted by one to three of NH₂, halogen, OH, CN or C₁₋₄alkylpiperazinyl; pyrimidinyl unsubstituted or substituted with NO₂, NH₂ or C₁₋₄alkyl; pyrazinyl unsubstituted or substituted with NO₂, NH₂ or C₁₋₄alkyl; thiazolyl unsubstituted or substituted with NO₂, NH₂ or C₁₋₄alkyl; benzimidazolyl unsubstituted or substituted with NO₂, NH₂ or C₁₋₄alkyl; pyrazolyl unsubstituted or substituted with NO₂, NH₂ or C₁₋₄alkyl; indazolyl unsubstituted or substituted with NO₂, NH₂ or C₁₋₄alkyl; quinolinyl unsubstituted or substituted with NO₂, NH₂ or C₁₋₄alkyl; indolyl unsubstituted or substituted with NO₂, NH₂ or C₁₋₄alkyl; -azaindazolyl unsubstituted or substituted with NO₂, NH₂ or C₁₋₄alkyl; deazapurinyl unsubstituted or substituted with NO₂, NH₂ or C₁₋₄alkyl; indanyl unsubstituted or substituted with NO₂, NH₂ or C₁₋₄alkyl; or morpholinoyl unsubstituted or substituted with NO₂, NH₂ or C₁₋₄alkyl.

In some embodiments, the compound of formula (I) is selected from:
4-(((3-chloro-1,4-dioxo-1,4-dihydronaphthalen-2-yl)amino)methyl)-N-hydroxybenzamide;
4-(((3-chloro-1,4-dioxo-1,4-dihydronaphthalen-2-yl)amino)methyl)-N-(pyridin-2-yl)benzamide;
N-(2-aminophenyl)-4-(((3-chloro-1,4-dioxo-1,4-dihydronaphthalen-2-yl)amino)methyl)benzamide;
4-(((3-chloro-1,4-dioxo-1,4-dihydronaphthalen-2-yl)amino)methyl)-N-(pyridin-3-yl)benzamide;
4-(((3-chloro-1,4-dioxo-1,4-dihydronaphthalen-2-yl)amino)methyl)-N-(pyridin-4-yl)benzamide;
4-(((3-chloro-1,4-dioxo-1,4-dihydronaphthalen-2-yl)amino)methyl)-N-(3-fluorophenyl)benzamide;
4-(((3-chloro-1,4-dioxo-1,4-dihydronaphthalen-2-yl)amino)methyl)-N-(4-fluorophenyl)benzamide;
4-(((3-chloro-1,4-dioxo-1,4-dihydronaphthalen-2-yl)amino)methyl)-N-phenylbenzamide;
4-(((3-chloro-1,4-dioxo-1,4-dihydronaphthalen-2-yl)amino)methyl)-N-(2-fluorophenyl)benzamide;
4-(((3-chloro-1,4-dioxo-1,4-dihydronaphthalen-2-yl)amino)methyl)-N-(thiazol-2-yl)benzamide;
N-(1H-benzo[d]imidazol-2-yl)-4-(((3-chloro-1,4-dioxo-1,4-dihydronaphthalen-2-yl)amino)methyl)benzamide;
4-(((3-chloro-1,4-dioxo-1,4-dihydronaphthalen-2-yl)amino)methyl)-N-(4-hydroxyphenyl)benzamide;
4-(((3-chloro-1,4-dioxo-1,4-dihydronaphthalen-2-yl)amino)methyl)-N-(3-ethynylphenyl)benzamide;
4-(((3-chloro-1,4-dioxo-1,4-dihydronaphthalen-2-yl)amino)methyl)-N-(2-fluoro-4-iodophenyl)benzamide;
N-(1H-benzo[d]imidazol-5-yl)-4-((3-chloro-1,4-dioxo-1,4-dihydronaphthalen-2-ylamino)methyl)benzamide;
4-((3-chloro-1,4-dioxo-1,4-dihydronaphthalen-2-ylamino)methyl)-N-cyclopropylbenzamide;
4-((3-chloro-1,4-dioxo-1,4-dihydronaphthalen-2-ylamino)methyl)-N-cyclopentylbenzamide;
4-((3-chloro-1,4-dioxo-1,4-dihydronaphthalen-2-ylamino)methyl)-N-(1H-indazol-5-yl)benzamide;
4-((3-chloro-1,4-dioxo-1,4-dihydronaphthalen-2-ylamino)methyl)-N-(5-methylthiazol-2-yl)benzamide;
4-((3-chloro-1,4-dioxo-1,4-dihydronaphthalen-2-ylamino)methyl)-N-(5-methyl-3H-pyrazol-3-yl)benzamide;
4-((3-chloro-1,4-dioxo-1,4-dihydronaphthalen-2-ylamino)methyl)-N-(3-nitropyridin-4-yl)benzamide;
4-((3-chloro-1,4-dioxo-1,4-dihydronaphthalen-2-ylamino)methyl)-N-(quinolin-6-yl)benzamide;
4-((3-chloro-1,4-dioxo-1,4-dihydronaphthalen-2-ylamino)methyl)-N-(quinolin-8-yl)benzamide;
4-((3-chloro-1,4-dioxo-1,4-dihydronaphthalen-2-ylamino)methyl)-N-(quinolin-3-yl)benzamide;
4-((3-chloro-1,4-dioxo-1,4-dihydronaphthalen-2-ylamino)methyl)-N-(quinolin-5-yl)benzamide;
4-((3-chloro-1,4-dioxo-1,4-dihydronaphthalen-2-ylamino)methyl)-N-(2-methylquinolin-4-yl)benzamide;
4-((3-chloro-1,4-dioxo-1,4-dihydronaphthalen-2-ylamino)methyl)-N-(1H-indol-5-yl)benzamide;
4-((3-chloro-1,4-dioxo-1,4-dihydronaphthalen-2-ylamino)methyl)-N-(2-methyl-1H-indol-5-yl)benzamide;
4-((3-chloro-1,4-dioxo-1,4-dihydronaphthalen-2-ylamino)methyl)-N-(1H-indol-7-yl)benzamide;
4-((3-chloro-1,4-dioxo-1,4-dihydronaphthalen-2-ylamino)methyl)-N-(1H-indol-4-yl)benzamide;
4-((3-chloro-1,4-dioxo-1,4-dihydronaphthalen-2-ylamino)methyl)-N-(4-(4-ethylpiperazin-1-yl)phenyl)benzamide;
4-((3-chloro-1,4-dioxo-1,4-dihydronaphthalen-2-ylamino)methyl)-N-(1H-indazol-6-yl)benzamide;
4-((3-chloro-1,4-dioxo-1,4-dihydronaphthalen-2-ylamino)methyl)-N-(1H-pyrrolo[2,3-b]pyridin-5-yl)benzamide;
4-((3-chloro-1,4-dioxo-1,4-dihydronaphthalen-2-ylamino)methyl)-N-(1H-pyrazolo[3,4-b]pyridin-5-yl)benzamide;
4-((3-chloro-1,4-dioxo-1,4-dihydronaphthalen-2-ylamino)methyl)-N-(7-methyl-7H-pyrrolo[2,3-d]pyrimidin-4-yl)benzamide;
4-((3-chloro-1,4-dioxo-1,4-dihydronaphthalen-2-ylamino)methyl)-N-(2,3-dihydro-1H-inden-4-yl)benzamide;
4-(((3-bromo-1,4-dioxo-1,4-dihydronaphthalen-2-yl)amino)methyl)-N-(pyridin-2-yl)benzamide;
4-(((3-chloro-1,4-dioxo-1,4-dihydronaphthalen-2-yl)amino)methyl)-N-(pyrimidin-4-yl)benzamide;
io 4-(((3-chloro-1,4-dioxo-1,4-dihydronaphthalen-2-yl)amino)methyl)-N-(pyrazin-2-yl)benzamide;
4-((3-chloro-1,4-dioxo-1,4-dihydronaphthalen-2-ylamino)methyl)-N-(pyridin-4-ylmethyl)benzamide;
4-((3-chloro-1,4-dioxo-1,4-dihydronaphthalen-2-ylamino)methyl)-N-(2-morpholinoethyl)benzamide;
N-(2-(1H-indol-3-yl)ethyl)-4-((3-chloro-1,4-dioxo-1,4-dihydronaphthalen-2-ylamino)methyl)benzamide;
4-(((3-chloro-1,4-dioxo-1,4-dihydronaphthalen-2-yl)amino)methyl)-N-(2-(dimethylamino)ethyl)benzamide;
4-(((3-chloro-1,4-dioxo-1,4-dihydronaphthalen-2-yl)amino)methyl)-N-(2-(pyrrolidin-1-yl)ethyl)benzamide;
4-(((3-chloro-1,4-dioxo-1,4-dihydronaphthalen-2-yl)amino)methyl)-N-(2-(diethylamino)ethyl)benzamide;
4-(((3-chloro-1,4-dioxo-1,4-dihydronaphthalen-2-yl)amino)methyl)-N-(2-(piperidin-1-yl)ethyl)benzamide; and
4-(((3-chloro-1,4-dioxo-1,4-dihydronaphthalen-2-yl)amino)methyl)-N-(2-(4-methylpiperazin-1-yl)ethyl)benzamide;
or a pharmaceutically acceptable salt, hydrate or isomer thereof.

In a further embodiment, the compound disclosed herein is 4-(((3-chloro-1,4-dioxo-1,4-dihydronaphthalen-2-yl)amino)methyl)-N-(pyridin-4-yl)benzamide, having the following formula:

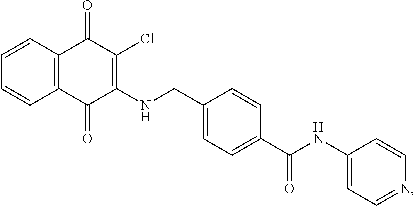 [SMILES: O=C(Nc1ccncc1)c1ccc(CNC2=C(Cl)C(=O)c3ccccc3C2=O)cc1]

or a pharmaceutically acceptable salt, hydrate or isomer thereof.

In some embodiments, the treatment and/or prevention of the optic neuropathy associated with apoptotic death of RGCs is via modulation of Nrf2 and TXNIP/NLRP3 inflammasome pathways in the retina.

In some embodiments, the compound disclosed herein enhances the expression of nuclear factor erythroid 2-related factor (Nrf2); downregulates thioredoxin interacting protein (TXNIP) expression, inhibits NLR family pyrin domain containing 3 (NLRP3) activation, and/or decreases inflammatory factors, interleukin (IL)-1P and IL-6 in the retina.

In some embodiments, the optic neuropathy associated with apoptotic death of RGCs is an optic nerve ischemic damage-related disease.

In some embodiments, the optic nerve ischemic damage-related disease is anterior ischemic optic neuropathy (AION) or posterior ischemic optic neuropathy (PION). In a further embodiment, the optic nerve ischemic damage-related disease is AION.

In some further embodiments, the AION is nonarteritic ischemic optic neuropathy (NAION) or arteritic anterior ischemic optic neuropathy (AAION). In some further embodiments, the PION is nonarteritic posterior ischemic optic neuropathy (NPION) or arteritic posterior ischemic optic neuropathy (APION).

In one embodiment, the compound described herein is administered subcutaneously.

### BRIEF DESCRIPTION OF THE DRAWINGS

The subject application file contains at least one drawing executed in color. Copies of this patent or patent application publication with color drawing(s) will be provided by the U.S. Patent Office upon request and payment of the necessary fee. The following drawings form part of the present specification and are included to further demonstrate certain aspects of the present disclosure, the inventions of which can be better understood by reference to one or more of these drawings in combination with the detailed description of specific embodiments presented herein. Exemplary embodiments are illustrated in referenced figures. It is intended that the embodiments and figures disclosed herein are to be considered illustrative rather than restrictive.

FIGS. 1 (A) to (C) show effects of M01 treatment on promoting RGCs survival in the AION Model. RGCs were retrogradely labeled by Fluoro-gold, and their densities at the center and mid-periphery retina were quantified from retinal flat mounts in sham, AION+PBS, AION+100 mg/Kg M01, and AION+200 mg/Kg groups (A). The survival rates of RGCs were 45.73%, 77.20%, and 58.87% at the central retina (B) and were 37.5%, 80.19%, and 63.82% at the mid-periphery retina (C) in AION+PBS, AION+100 mg/Kg M01, and AION+200 mg/Kg groups, respectively. n=6 in each group. *, p<0.05, **, p<0.01, ****, p<0.0001. Scale bar=50|im.

FIG. 2 shows effects of M01 treatment on preserving visual function in AION Models. The P1-N2 amplitudes of the sham and the AION+100 mg/Kg M01 groups were considerably higher than those of the AION+PBS group, according to the FVEP data. However, no statistically significant change in P1-N2 amplitudes exists between the AION+PBS. n=6 in each group. *, p<0.05, ***, p<0.001.

FIGS. 3 (A) and (B) show that M01 treatment mitigated ischemia-induced RGC apoptosis. RGCs apoptosis was assessed by TUNEL (green), and the nuclei were labeled with DAPI (blue) (A). TUNEL-positive cells at the GCL layer of the retina were greatly enhanced by AION induction, while M01 therapy efficiently reduced TUNEL-positive cells at the GCL layer of the retina. n=6 in each group. **, p<0.01, ****, p<0.0001. Scale bar=50|im (B).

FIGS. 4 (A) and (B) show that M01 treatment reduced ischemia-induced macrophage infiltration in ON. The anti-ED-1 (green) antibody was used to detect macrophage infiltration in ON, while the DAPI (blue) was utilized to identify nuclei (A). The number of ED-1positive cells in the ON increased after ON infarction. However, the M01 treatment reduced the amount of ED-1-positive cells in the ON. *, p<0.05, ***, p<0.001. Scale bar=100 ym at the upper column and 50|im at the lower column (B).

FIGS. 5 (A) to (H) show that M01 decreased microglial infiltration in the retina and modulated microglial polarization in the AION model. (A), (C), (E) Immunofluorescent images and the quantitative data of Iba1(B) and IL-6 (D) expression in the retina and Ym1 (F) expression in the ON. (G) Immunoblotting image and quantification of Argi (H) protein expression levels and IL-ip (I) in the retina. M01 therapy significantly reduced ischemia-induced Iba1-positive cells and pro-inflammatory cytokines, IL1p and IL-6, in the retina while significantly increasing M2 markers, Ym1, and Arg1 expression after ON ischemic injury. n=6 in each group. *, p<005, **, p<0.01, ***, p<0.001. The scale bar at A and C=50 lim. The scale bar at E=100 ym at the upper column and 50 ym at the lower column.

FIGS. 6 (A) and (B) shows that M01 treatment alleviated ischemia-induced ONH edema. Image-guided OCT was used in each group to detect ONH edema. The OCT scans showed that ON infarction caused persistent disc edema (A), and M01 treatment significantly alleviated disc edema 28 days after AION induction. n=6 in each group; p<0.05 (B).

FIG. 7 shows that M01 treatment attenuated ischemia-induced ON demyelinatio n. Confocal images showed markedly reduced CNPase levels in the ON after ON infarction, while the M01 treatment retained the myelination of ON. ON longitudinal sections were stained with antibodies against CNPase (green), and nuclei were stained with DAPI (blue). n=6 in each group. *, p<0.05, ****, p<0.0001. Scale bars=100|im at the upper column; 50|im at the lower column.

FIG. 8 shows that M01 modulated Nrf2/TXNIP/NLRP3 axis following ON infarction. (A) The immunoblotting analysis showed Nrf2, NEDD4, TXNIP, and NLRP3 protein expression levels in sham, AION+PBS, and AION+M01 groups. (B-D) The quantitative data of (A). GAPDH was used as an internal loading control. All data are shown as mean±S.D; n=6 in each group.

### DETAILED DESCRIPTION OF THE INVENTION

The invention is, at least in part, based on the discovery of using aminonaphthoquinone compounds and their effective dose in the prevention and/or treatment of an optic neuropathy associated with apoptotic death of retinal ganglion cells (RGCs).

As used herein, except where the context requires otherwise, the term “comprise” and variations of the term, such as “comprising,” “comprises” and “comprised” are not intended to exclude other additives, components, integers or steps.

As used herein, except where the context requires otherwise, the method steps disclosed are not intended to be limiting nor are they intended to indicate that each step is essential to the method or that each step must occur in the order disclosed.

As used herein, the use of “or” means “and/or” unless stated otherwise. In the context of a multiple dependent claim, the use of “or” refers back to more than one preceding independent or dependent claim in the alternative only.

As used herein, all numbers are approximate, and may be varied to account for measurement error and the rounding of significant digits. The use of “about” before certain measured quantities includes variations due to sample impurities, measurement error, human error, and statistical variation, as well as the rounding of significant digits.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal and ammonium salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like.

The terms “administer,” “administering,” or “administration,” as used herein, refers to implanting, absorbing, ingesting, injecting, inhaling, or otherwise introducing an inventive compound, or a pharmaceutical composition thereof, in or on a subject.

As used herein, the terms “condition,” “disease,” and “disorder” are used interchangeably.

An “effective amount” of a compound described herein refers to an amount sufficient to elicit the desired biological response, i.e., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent.

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

As used herein, the term “pharmaceutically acceptable carrier” refers to a solid, semisolid, or liquid filler, diluent, encapsulating material, formulation auxiliary, or carrier conventional in the art for use with a therapeutic agent for administration to a subject. A pharmaceutically acceptable carrier is non-toxic to recipients at the dosages and concentrations employed and is compatible with other ingredients of the formulation. Pharmaceutically acceptable carriers are determined in part by the particular composition being administered, as well as by the particular method used to administer the composition.

As used herein, the term “subject” is defined herein to include animals such as mammals, including, but not limited to, primates (e.g., humans), cows, sheep, goats, horses, dogs, cats, rabbits, rats, mice and the like. In specific embodiments, the subject is a human. The terms “subject” and “patient” are used interchangeably herein in reference, for example, to a mammalian subject, such as a human.

As used herein, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or dosage form provided herein, with or without one or more additional active agents, after the diagnosis or onset of symptoms of the particular disease.

As used herein, the terms “prevent,” “preventing” and “prevention” refer to the prevention of the onset, recurrence or spread of a disease or disorder, or of one or more symptoms thereof. In certain embodiments, the terms refer to the treatment with or administration of a compound or an antibody or dosage form provided herein, with or without one or more other additional active agents, prior to the onset of symptoms, particularly to patients at risk of disease or disorders provided herein. The terms encompass the inhibition or reduction of a symptom of the particular disease. In this regard, the term “prevention” may be interchangeably used with the term “prophylactic treatment.”

As used herein, the terms “co-administration” and “in combination with” include the administration of two or more therapeutic agents simultaneously, concurrently, separately or sequentially within no specific time limits unless otherwise indicated. In one embodiment, the therapeutic agents are in the same composition or unit dosage form. In other embodiments, the therapeutic agents are in separate compositions or unit dosage forms.

Apoptotic death of retinal ganglion cells (RGCs) is a common pathologic feature in different types of optic neuropathy, such as ischemic optic neuropathy and glaucoma, and will lead to irreversible loss of visual function. Potent and effective protection against RGC death is determinative in developing a successful treatment for these optic neuropathies. The disclosure evaluates the neuroprotective effect of a HECT domain-E3 ubiquitin ligase inhibitor, the compounds described herein, on retinal ganglion cells after ischemic injury. Administration of the compounds described herein significantly increase RGC survival and preserved visual function after AION induction. The number of TUNEL-positive cells and ED1-positive cells are significantly decreased, and optic disc edema is reduced considerably after ischemic infarction with the compound treatment. Moreover, the compounds described herein effectively ameliorate optic nerve demyelination and enhanced M2 microglial polarization after AION induction. The compounds described herein enhance the expression of nuclear factor erythroid 2-related factor (Nrf2); downregulate Thioredoxin interacting protein (TXNIP) expression, inhibit NLR family pyrin domain containing 3 (NLRP3) activation, and further decrease inflammatory factors, interleukin (IL)-1P and IL-6 in the retina after ischemic injury. These findings suggested that the compounds described herein have therapeutic potential in optic nerve ischemic damage-related diseases by modulating Nrf2 and TXNIP/NLRP3 inflammasome pathways in the retina.

The compounds described herein can be prepared using methods known to those skilled in the art in view of this disclosure. The compounds and their preparation methods described herein have been disclosed in PCT/US2015/041767 and U.S. Ser. No. 62/199,207. For example, the preferred compounds of the invention can be prepared as shown in the following schemes:

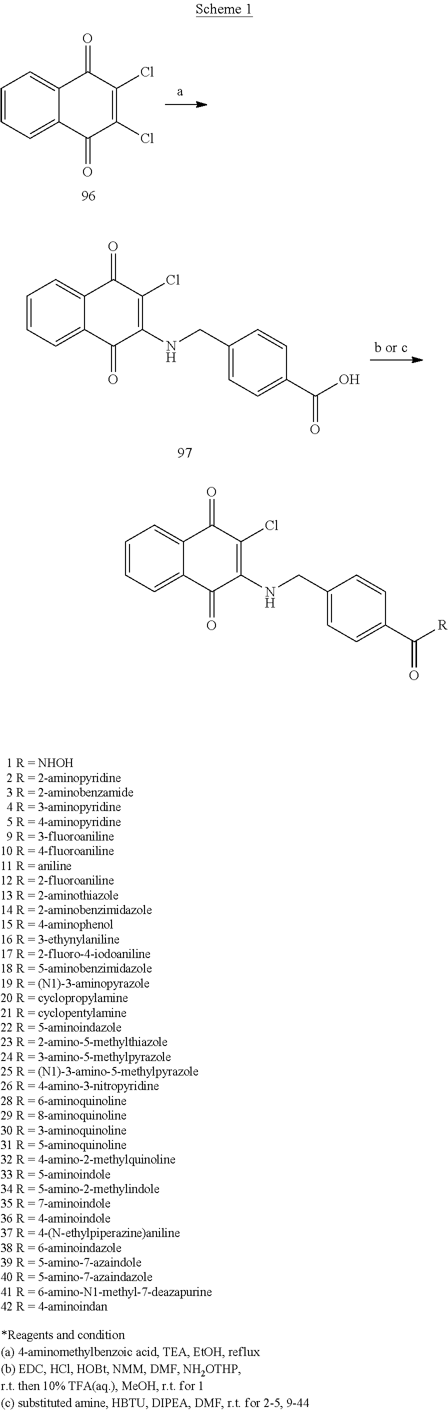

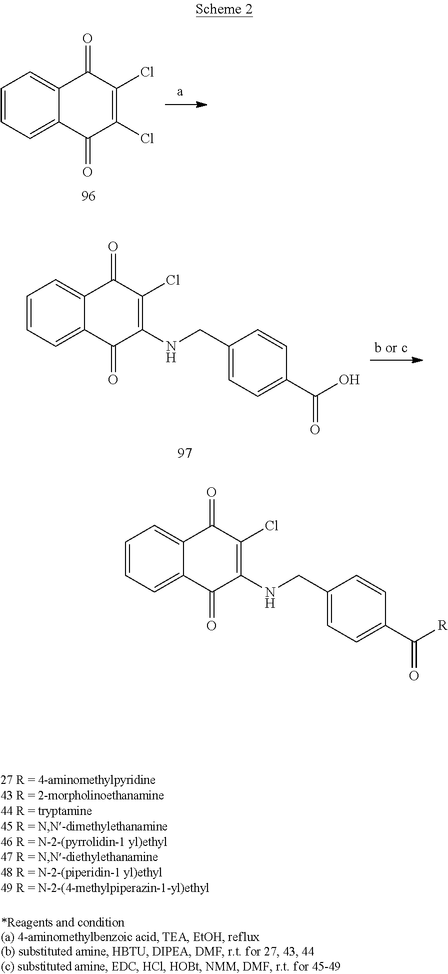

In another aspect, the compound described herein is in a form of a pharmaceutical composition or combination comprising a compound of Formula (I) or a pharmaceutically acceptable salt, hydrae or isomer as an active ingredient. In one embodiment, the pharmaceutical composition or combination is in one or more unit dosage forms. Preferably, the dosage form is in one or more capsule forms or tablet forms. In one embodiment, the compound of Formula (I) is 4-(((3-chloro-1,4-dioxo-1,4-dihydronaphthalen-2-yl)amino)methyl)-N-(pyridin-3-yl)benzamide.

In some embodiments, the pharmaceutical composition of the invention comprises about 100 mg to about 300 mg, about 150 mg to about 300 mg, about 150 mg to about 250 mg, about 200 mg to 250 mg, about 220 mg to about 280 mg, about 220 mg to about 250 mg or about 200 mg to about 220 mg of the active ingredient in a single tablet; preferably, about 200 mg or 220 mg in a single tablet. In another further embodiment, the pharmaceutical composition of the invention comprises about 100 mg to about 500 mg, about 150 mg to about 500 mg, about 180 mg to about 500 mg, about 200 mg to about 500 mg, about 150 mg to about 350 mg, about 150 mg to about 300 mg, about 200 mg to about 400 mg, about 200 mg to about 400 about 350 mg, about 200 mg to about 300 mg, about 250 mg to about 500 mg, about 250 mg to about 400 mg, about 250 mg to about 350 mg or about 250 mg to about 300 mg of the active ingredient in a single capsule; preferably, about 250 mg of the active ingredient in a single capsule.

While it may be possible for the compounds of the invention to be administered as the raw chemical, it is also possible to present them as a pharmaceutical formulation. Accordingly, the present invention provides a pharmaceutical formulation or composition comprising a compound or a pharmaceutically acceptable salt, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Formulations may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Remington: The Science and Practice of Pharmacy, A. Gennaro, ed., 20th edition, Lippincott, Williams & Wilkins, Philadelphia, Pa. Suitable liquid carriers, especially for injectable solutions, include water, aqueous saline solution, aqueous dextrose solution, and glycols. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration, although the most suitable route may depend, for example, upon the condition and disorder of the recipient. Oral administration is a preferred route. The formulations may conveniently be presented in unit dosage form and may be prepared by any of the methods well known in the art of pharmacy. All methods include the step of bringing into association a compound of the present invention or a pharmaceutically acceptable salt, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

For oral administration, suitable pharmaceutical compositions of the invention include powders, granules, pills, tablets, lozenges, chews, gels, and capsules as well as liquids, syrups, suspensions, elixirs, and emulsions. These compositions may also include anti-oxidants, flavorants, preservatives, suspending, thickening and emulsifying agents, colorants, flavoring agents and other pharmaceutically acceptable additives. Formulations for oral administration may be formulated to be immediate release or modified release, where modified release includes delayed, sustained, pulsed, controlled, targeted and programmed release.

For parenteral administration, the compounds of the present invention are administered directly into the blood stream, into muscle, or into an internal organ via an intravenous, intraarterial, intraperitoneal, intramuscular, subcutaneous or other injection or infusion. Parenteral formulations may be prepared in aqueous injection solutions which may contain, in addition to the compound of the invention, buffers, antioxidants, bacteriostats, salts, carbohydrates, and other additives commonly employed in such solutions. Parenteral administrations may be immediate release or modified release (such as an injected or implanted depot).

Compounds or compositions of the present invention may also be administered topically, (intra)dermally, or transdermally to the skin or mucosa. Typical formulations include gels, hydrogels, lotions, solutions, creams, ointments, dressings, foams, skin patches, wafers, implants and microemulsions. Compounds or compositions of the present invention may also be administered via inhalation or intranasal administration, such as with a dry powder, an aerosol spray or as drops. Additional routes of administration for compounds of the present invention include intravaginal and rectal (by means of a suppository, pessary or enema), and ocular and aural.

Dosage regimens can be adjusted to provide the optimum therapeutic response. For example, several divided dosages may be administered daily or the dosage may be proportionally reduced as indicated by the exigencies of the therapeutic situation.

It is especially advantageous to formulate the compounds in dosage unit form for ease of administration and uniformity of dosage. Dosage unit form as used herein refers to physically discrete units suited as unitary dosages for the subjects to be treated; each containing a therapeutically effective quantity of the compound and at least one pharmaceutical excipient. A drug product will comprise a dosage unit form within a container that is labelled or accompanied by a label indicating the intended method of treatment.

It is understood that the examples described herein are merely illustrative of the present invention. Certain modifications of the articles and/or methods employed may be made and still achieve the objectives of the invention. Such modifications are contemplated as within the scope of the claimed invention.

### Example

### Materials and Methods

### Animals

Outbred adult male Wistar rats weighing 100-125 grams (4-6 weeks) were procured from BioLASCO Co. in Taiwan and housed in filter-top holding cages at Tzu Chi University's Laboratory Animal Center. Tzu Chi University's Institutional Animal Care and Use Committee (IACUC) (No. 109069, 04/15/2021) authorized animal care and experimental protocols. For general anesthesia, a ketamine (100 mg/kg) and xylazine (10 mg/kg) cocktail was delivered intramuscularly. In all trials, 0.5% Alcaine (Alcon, Puurs, Belgium) was used for local anesthetic, while Mydrin-P (Santen Pharmaceutical Co., Ltd., Osaka, Japan) was used for pupil dilation.

### Experimental Ischemic Optic Neuropathy Rat Model (AION Induction)

The photodynamic thrombosis approach created an experimental ischemic optic neuropathy model. 2.5 mM rose bengal in pH 7.4 PBS (1 mL/kg animal weight) was injected into the rat tail vein. The optic disc was subjected to an argon green laser (514 nm wavelength, 500 mm size, and 80 mW power) for a total of 12 pulses (one pulse per second) immediately after injection (within one minute).

### Electroretinography (ERG) and Flash Visual Evoked Potential (FVEP)

The full-filed flash ERG and FVEP [13]were performed 28 days after the ischemic infarction utilizing a visual electrodiagnostic system (CELERIS System; Diagnosys LLC, MA, USA) 28 days after the isch emic infarct. The primary visual cortex electrodes were considered active (positive) electrodes, the frontal cortex electrode was considered the reference (negative) electrode, and the electrode at the tail served as the ground electrode. For FVEP, the parameters were at a flash intensity of 0.5 cd·s/m2, a flash frequency of 1.02 Hz with no background Illumination, and an intensity of 10 cd·s/m2 for photopic negative response (PhNR). An average of 100 sweeps were collected, and the P1-N2 amplitude of FVEP was recorded to evaluate the integrity of visual function. PhNR amplitude was measured from baseline to negative trough following b-wave to evaluate RGC and inner retina (amacrine cells and Muller-glial cells) function.

### Image-Guided OCT Imaging

As previously described [8], image-guided OCT imaging was carried out. In brief, the image of Bruch membrane opening minimum rim width (represented as optic nerve width (ONW)) was taken on day 1, day 3, day 7, day 14, and day 28 post-AION. At least six clear captures were obtained for each eye. The average ONW was calculated and analyzed using GraphPad Prism 7.0.

### Retrograde Labeling of RGCs by Fluoro-Gold and Measurement of RGC Density

RGCs were tagged retrogradely, as previously described [35]. In brief, one week before the animals were euthanized, retrograde labeling was conducted to avoid overcounting the macrophage or microglial cells. The eyecups were extracted and preserved in 10% formalin one week after labeling. Under a fluorescent microscope with 100× power, the retina was flat mounted and studied from 1 mm to 3 mm from the optic disc to calculate central and peripheral. The retina was examined from 1 mm to 3 mm from the center to calculate central and peripheral RGC densities. At least ten random locations were counted independently, and photos were processed using Image J software.

### Immunohistochemistry (IHC) on ON and Retina Tissues

IHC was used to identify a set of markers suggesting the processes of neuroinflammation, autophagy, and microglia polarization to evaluate the influence of an E3 ligase inhibitor (compound M01) on the structure and morphology of retinal and optic nerve following ischemia injury. Detailed processes have already been described[8]. In brief, retina or ON slices were blocked in blocking buffer before being incubated overnight at four ° C. with primary antibodies against ED1, Iba1, IL-6, Ym-1, and CNPase, diluted in antibody dilution buffer (1% BSA, 0.1% cold fish skin gelatin, and 0.5% Triton X-100 in 1 PBS (pH 7.2); 1:200). Secondary antibodies were added to the tissues and incubated at room temperature for 1 hour. The photographs were taken with a Zeiss confocal laser-scanning microscope (Carl Zeiss, Inc).

### TUNEL Assay

Apoptosis of retinal ganglion cells resulting in axon degeneration is considered an early pathologic change in ischemic optic neuropathy. Apoptotic cells in the ganglion cell layer (GCL) were visualized by TUNEL assay (DeadEnd™ Fluorometric TUNEL System; Promega Corporation, WI, USA) and followed the manufacturer's instructions. Retina sections were examined with a fluorescent microscope (Zeiss), and TUNEL+cells in the GCL were counted by Image J software. Apoptotic cells were quantified by measuring TUNEL-positive cells from the entire section, and the average number of apoptotic cells per section was calculated from 6 sections of both eyes of 3 rats from each group.

### Immunoblotting Analysis

Immunoblotting was performed as previously described. Fifty (ig of protein was separated on a 10% SDS gel, transferred to polyvinylidene difluoride (PVDF) membranes, and the membrane was incubated with the primary antibodies against Iba1, IL-6, IL-1B, NLRP3, TXNIP, NEDD4, Argi and GAPDH (1:2000; Sigma-Aldrich co, MO). The membranes were further incubated with the appropriate secondary antibodies. The images were taken and analyzed using the ChemiDoc MP system (Bio-Rad Laboratories, CA, USA).

### Statistical Analysis

All data were given as the mean±S.D. We performed statistical analysis using GraphPad Prism 7.0 (GraphPad Software, La Jolla, CA, USA). We used the Kruskal-Wallis test followed by Dunn's multiple comparison test adjustments for comparing more than two groups. P-values less than 0.05 was considered statistically significant, with * denoting p<0.05, ** p<0.01, and *** p<0.001.

### Example 1 M01 Promoted RGC Survival Post-Infarction

RGCs were retrogradely labeled with Fluoro-gold, and retinal wholemount was conducted to evaluate RGC survival following M01 treatment after AION induction. M01 is 4-(((3-chloro-1,4-dioxo-1,4-dihydronaphthalen-2-yl)amino)methyl)-N-(pyridin-4-yl)benzamide, having the following formula:

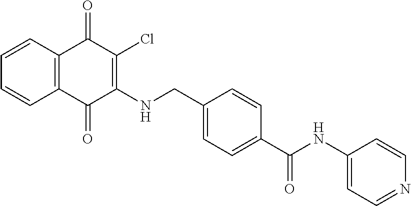 [SMILES: O=C(Nc1ccncc1)c1ccc(CNC2=C(Cl)C(=O)c3ccccc3C2=O)cc1]

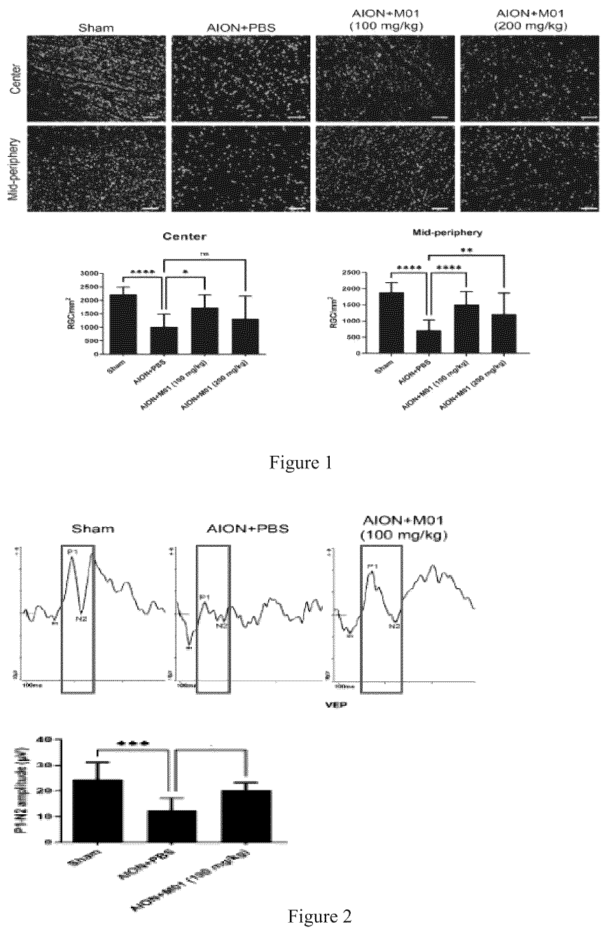

The RGCs in the central and mid-peripheral retina (FIG. 1) were significantly conserved four weeks after the infarction in the 100 mg/Kg M01-treated group compared to the PBS-treated group. 100 mg/Kg M01 treatment increased the RGC survival rate by 31.47% in the central (Sham:2237±257 cells/mm2; AION+PBS: 1023±458 cells/mm2; AION+100 mg/Kg M01:1727±485 cells/mm²; AION+200 mg/Kg M01:1317±846 cells/mm²) and 42.46% in the mid-peripheral retina (Sham:1888±295 cells/mm2; AION+PBS: 708±322 cells/mm²; AION+100 mg/Kg M01:1514±391 cells/mm²; AION+200 mg/Kg M01:1205±662 cells/mm²).

### Example 2 M01 Preserved Visual Function Post-Infarction

FVEP was recorded to monitor visual function. The P1-N2 amplitudes decreased significantly after AION induction compared to the sham group (FIG. 2), while the 100 mg/Kg M01 treatment effectively preserved visual function at 28 days postinfarction (Sham: 24.29±6.81 yV; AION+PBS: 12.34±4.88 yV; AION+100 mg/Kg M01:20.10±3.09 yV; AION+200 mg/Kg M01: 19.57±5.38 yV). The 100 mg/Kg M01 therapy considerably increased the RGC survival rate and visual function based on RGC density and FVEP measurement assessment. M01 at 100 mg/Kg was chosen for further processing.

### Example 3 M01 Reduced RGC Apoptosis Post-Infarction

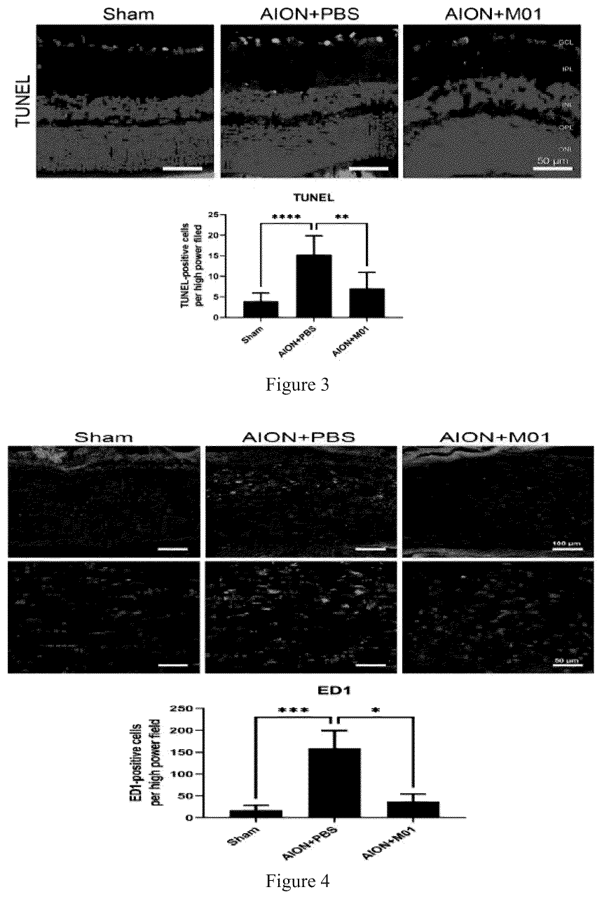

Frozen retinal sections were prepared for TUENL experiments and examined by confocal microscopy to determine if M01 reduces apoptotic RGC death following ischemic injury. FIG. 3 showed that the mean number of TUNEL-positive cells in the PBS-injected retinas was significantly higher than in the sham retinas (15.3±4.5/HPF vs. 4.0±2.0/HPF, n=6, p<0.001), whereas M01 treatment significantly reduced the mean number of TUNEL-positive cells in the retina (7.1±3.9/HPF vs. AION+PBS, n=6, p=0.0019). This finding suggests that M01 therapy effectively reduced the photothrombotic impact on the optic nerve.

### Example 4 M01 Alleviated Microglial Infiltration in the ON and Modulated Microglial Polarization Post-Infarction

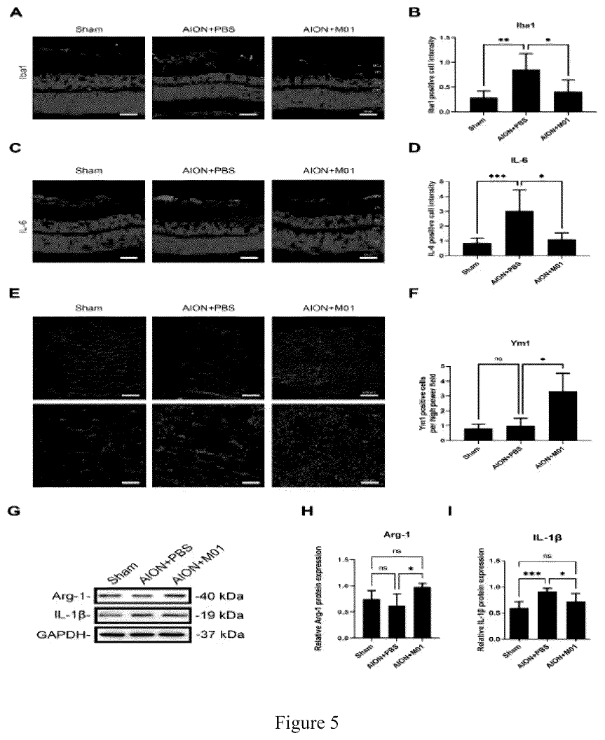

In the pathogenesis of ischemic optic neuropathy, activated macrophages or microglia can trigger apoptotic death of RGCs. We discovered ED1- (FIG. 4) and Ibal-positive cells (FIGS. 5A and 5B) infiltrating the optic nerve and retina 28 days after an ON infarction, indicating that microglial activation is strongly regulated in the PBS-treated group compared to the sham group (ED-1: AION+PBS: 160.0±39.6/HPF, Sham: 17.5±11.3/HPF, n=6, p<0.001). Compared to PBS treatment, M01 therapy reduced ED-positive and Ibal-positive cell infiltration in the ON and retina (ED-1: AION+M01:36.9±17.6/HPF vs. AION+PBS, n=6, p=0.0419). Furthermore, IHC and immunoblotting results show that the PBS-treated group had significantly higher M1 microglia markers, IL-6 (FIG. 5C and FIG. 5D) and IL-13 (FIGS. 5G and 5I), than the sham group. In contrast, the M2 microglia markers, Ym1 (FIGS. 5E and 5F) and Arg-1(FIGS. 5G and 5H) were upregulated in the M01-treated group compared with the PBS-treated group. Those results suggest that M01 treatment promoted M2 polarization and minimized inflammatory response through markedly reduced M1 microglia activation and pro-inflammatory cytokines secretion.

### Example 5 Treatment with M01 attenuated ONH edema post-infarction

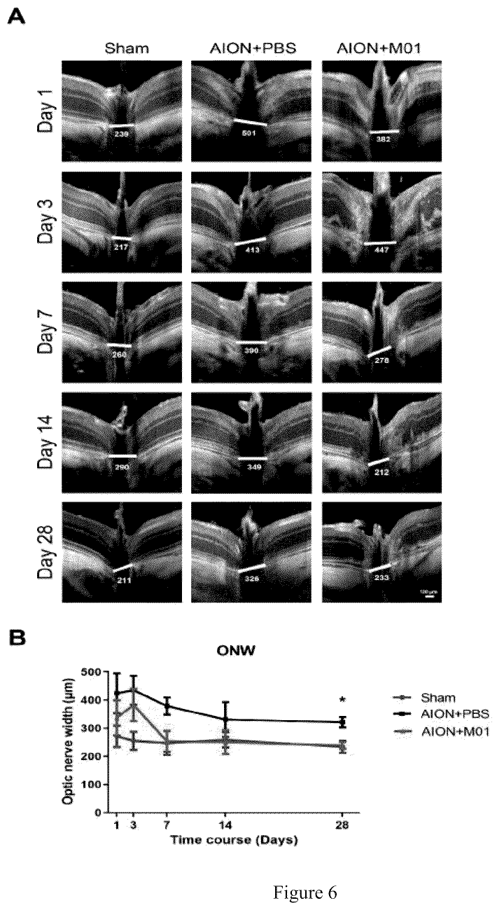

Optic disc swelling is one of the most common clinical features in ischemic optic neuropathy. The spectral-domain OCT was performed to assess the effects of M01 treatment on the rAION model to monitor ONH width at day 1, 3, 7, 14, and day 28 post-AION (FIG. 6). Four weeks after AION induction and M01 treatment, there was a significant attenuation of ONH edema in the M01-treated AION eyes compared with PBS-treated AION eyes (AION+PBS:321.26±18.60 ym, n=6; AION+M01: 238.82±13.00 ym, n=6; p<0.05).

### Example 6 Treatment with M01 Maintained Myelination of ON Post-Infarction

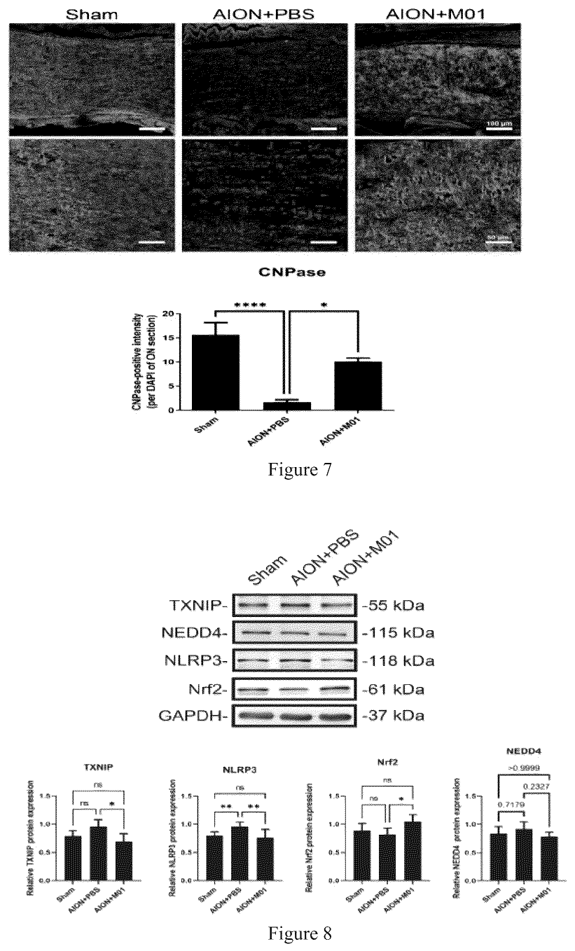

Myelination is essential for protecting and supporting ON fibers and is responsible for the efficient transmission of visual signals along the nerve fibers. Optic nerve demyelination can develop secondary to the ischemic insult because the obstruction or decreased blood flow might disturb the feeding of the myelin-producing cells, resulting in myelin breakdown [36-38]. Therefore, after ischemia damage, we examined the levels of 23-cyclic nucleotide 3iphosphodiesterase (CNPase), the primary myelin-associated enzyme in the CNS, in the optic nerve. CNPase levels were considerably lower in the PBS-treated group compared to the sham group (FIG. 7). M01 administration prevents the ON's CNPase level from dropping dramatically following AION induction. These findings imply that MOI therapy preserved the integrity of ON myelination after an ischemic insult.

### Example 7 M01 Induced Nrf2 Signaling and Suppressed TXNIP/NLRP3 Activation after AION Induction

Nrf2 activation reduced oxidative damage to RGCs, regulated microglial polarization, and attenuated the ON and retina inflammatory response, preserving visual function after ON ischemic injury. Moreover, NEDD4 has been demonstrated to be an E3 ubiquitin ligase of Nrf2 and to facilitate Nrf2 ubiquitination. NEDD4 inhibition alleviated oxidative stress by reducing Nrf2 degradation. The protein levels of Nrf2, NEDD4, TXNIP, and NLRP3 were assessed using immunoblotting to understand further the molecular mechanism underlying M01's protective actions on RGCs. According to immunoblotting data, TXNIP and NLRP3 expression levels were considerably higher in the AION+PBS group than in the sham group. M01 therapy might increase Nrf2 expression while decreasing TXNIP and NLRP3 expression in the retina after AION induction (FIG. 8). There is no significant difference in NEDD4 expression level among the sham, AION+PBS, and AION+M01 groups. These findings suggested that M01 protected RGCs following ON ischemia damage by inhibiting TXNIP and NLRP3 activation via Nrf2 upregulation. Furthermore, M01 showed no inhibiting effect on NEDD4 expression in the retina.

What is claimed is:

1. A method for preventing and/or treating an optic neuropathy associated with apoptotic death of RGCs, comprising administering an effective amount of a compound of Formula (I) or a pharmaceutically acceptable salt, hydrate or isomer as an active ingredient to the subject, 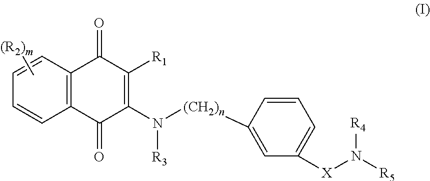
wherein
R₁ is halogen;
each R₂ is the same or different, representing H, C₁₋₁₀alkyl, C₂₋₁₀alkenyl, C₂₋₁₀alkynyl, NH₂, NO₂, C₁₋₁₀alkyloxy, C₁₋₁₀alkylthio, C₁₋₁₀alkylamino, C₁₋₁₀alkyloxyC₁₋₁₀alkyl, OH or CN, C₆₋₁₀aryl or C₅₋₇heterocyclic having 1 to 3 heteroatoms selected from the group consisting of N, O and S;
R₃ is H, C₁₋₁₀alkyl, C₂₋₁₀alkenyl, C₂₋₁₀alkynyl, NH₂, NO₂, OH or CN;
R₄ is H, C₁₋₁₀alkyl, C₂₋₁₀alkenyl, C₂₋₁₀alkynyl, NH₂, NO₂, OH or CN;
R₅ is OH, C₃₋₈cycloalkyl, phenyl unsubstituted or substituted with one to three same or different substituents selected from OH, CN, halogen, NH₂ or C₁₋₄alkylpiperazinyl, C₁₋₆alkylpiperazinyl, C₁₋₆alkylpyridinyl, C₁₋₆alkylpyrrolidinyl, pyridinyl, pyrimidinyl, pyrazinyl. piperazinyl, pyrrolidinyl, thiazolyl, benzimidazolyl, pyrazolyl, indazolyl, quinolinyl, indolyl, azaindolyl, azaindazolyl, deazapurinyl, indanyl, morpholinoyl or C₁₋₄ alkylmorpholinoyl, each of which is unsubstituted or substituted with one, two or three groups selected from OH, CN, halogen, NO₂, C₁₋₄alkyl, or NH₂;
X is —C(O)
Y is —N—;
m is an integer of 0-3; and
n is an integer of 1-7.

2. The method of claim 1, wherein m is 0; R₁ is halogen; n is any integer of 1-4; R₃ is H; X is C(O); R₄ is H; and R₅ is OH, C₃₋₈cycloalkyl, phenyl unsubstituted or substituted with one to three same or different substituents selected from OH, CN, halogen, NH₂ or C₁₋₄alkylpiperazinyl, C₁₋₆alkylpiperazinyl, C₁₋₆alkylpyridinyl, C₁₋₆alkylpyrrolidinyl, pyridinyl, pyrimidinyl, pyrazinyl. piperazinyl, pyrrolidinyl, thiazolyl, benzimidazolyl, pyrazolyl, indazolyl, quinolinyl, indolyl, io azaindolyl, azaindazolyl, deazapurinyl, indanyl, morpholinoyl or C₁₋₄alkylmorpholinoyl, each of which is unsubstituted or substituted with one, two or three groups selected from OH, CN, halogen or NH₂.

3. The method of claim 1, wherein m is 0; R₁ is halogen; n is any integer of 1-2; R₃ is H; X is C(O); R₄ is H; and R₅ is OH, C₃₋₈cycloalkyl, pyridinyl, phenyl substituted by one to three of is NH₂, halogen, OH, CN or C₁₋₄alkylpiperazinyl; pyrimidinyl unsubstituted or substituted with NO₂, NH₂ or C₁₋₄alkyl; pyrazinyl unsubstituted or substituted with NO₂, NH₂ or C₁₋₄alkyl; thiazolyl unsubstituted or substituted with NO₂, NH₂ or C₁₋₄alkyl; benzimidazolyl unsubstituted or substituted with NO₂, NH₂ or C₁₋₄alkyl; pyrazolyl unsubstituted or substituted with NO₂, NH₂ or C₁₋₄alkyl; indazolyl unsubstituted or substituted with NO₂, NH₂ or C₁₋₄alkyl; quinolinyl unsubstituted or substituted with NO₂, NH₂ or C₁₋₄alkyl; indolyl unsubstituted or substituted with NO₂, NH₂ or C₁₋₄alkyl; -azaindazolyl unsubstituted or substituted with NO₂, NH₂ or C₁₋₄alkyl; deazapurinyl unsubstituted or substituted with NO₂, NH₂ or C₁₋₄alkyl; indanyl unsubstituted or substituted with NO₂, NH₂ or C₁₋₄alkyl; or morpholinoyl unsubstituted or substituted with NO₂, NH₂ or C₁₋₄alkyl.

4. The method of claim 1, wherein the the compound of formula (I) is selected from:
4-(((3-chloro-1,4-dioxo-1,4-dihydronaphthalen-2-yl)amino)methyl)-N-hydroxybenzamide;
4-(((3-chloro-1,4-dioxo-1,4-dihydronaphthalen-2-yl)amino)methyl)-N-(pyridin-2-yl)benzamide;
N-(2-aminophenyl)-4-(((3-chloro-1,4-dioxo-1,4-dihydronaphthalen-2-yl)amino)methyl)benzamide;
4-(((3-chloro-1,4-dioxo-1,4-dihydronaphthalen-2-yl)amino)methyl)-N-(pyridin-3-yl)benzamide;
4-(((3-chloro-1,4-dioxo-1,4-dihydronaphthalen-2-yl)amino)methyl)-N-(pyridin-4-yl)benzamide;
4-(((3-chloro-1,4-dioxo-1,4-dihydronaphthalen-2-yl)amino)methyl)-N-(3-fluorophenyl)benzamide;
4-(((3-chloro-1,4-dioxo-1,4-dihydronaphthalen-2-yl)amino)methyl)-N-(4-fluorophenyl)benzamide;
4-(((3-chloro-1,4-dioxo-1,4-dihydronaphthalen-2-yl)amino)methyl)-N-phenylbenzamide;
4-(((3-chloro-1,4-dioxo-1,4-dihydronaphthalen-2-yl)amino)methyl)-N-(2-fluorophenyl)benzamide;
4-(((3-chloro-1,4-dioxo-1,4-dihydronaphthalen-2-yl)amino)methyl)-N-(thiazol-2-yl)benzamide;
N-(1H-benzo[d]imidazol-2-yl)-4-(((3-chloro-1,4-dioxo-1,4-dihydronaphthalen-2-yl)amino)methyl)benzamide;
4-(((3-chloro-1,4-dioxo-1,4-dihydronaphthalen-2-yl)amino)methyl)-N-(4-hydroxyphenyl)benzamide;
4-(((3-chloro-1,4-dioxo-1,4-dihydronaphthalen-2-yl)amino)methyl)-N-(3-ethynylphenyl)benzamide;
4-(((3-chloro-1,4-dioxo-1,4-dihydronaphthalen-2-yl)amino)methyl)-N-(2-fluoro-4-iodophenyl)benzamide;
N-(1H-benzo[d]imidazol-5-yl)-4-((3-chloro-1,4-dioxo-1,4-dihydronaphthalen-2-ylamino)methyl)benzamide;
4-((3-chloro-1,4-dioxo-1,4-dihydronaphthalen-2-ylamino)methyl)-N-cyclopropylbenzamide;
4-((3-chloro-1,4-dioxo-1,4-dihydronaphthalen-2-ylamino)methyl)-N-cyclopentylbenzamide;
4-((3-chloro-1,4-dioxo-1,4-dihydronaphthalen-2-ylamino)methyl)-N-(1H-indazol-5-yl)benzamide;
4-((3-chloro-1,4-dioxo-1,4-dihydronaphthalen-2-ylamino)methyl)-N-(5-methylthiazol-2-yl)benzamide;
4-((3-chloro-1,4-dioxo-1,4-dihydronaphthalen-2-ylamino)methyl)-N-(5-methyl-3H-pyrazol-3-yl)benzamide;
4-((3-chloro-1,4-dioxo-1,4-dihydronaphthalen-2-ylamino)methyl)-N-(3-nitropyridin-4-yl)benzamide;
4-((3-chloro-1,4-dioxo-1,4-dihydronaphthalen-2-ylamino)methyl)-N-(quinolin-6-yl)benzamide;
4-((3-chloro-1,4-dioxo-1,4-dihydronaphthalen-2-ylamino)methyl)-N-(quinolin-8-yl)benzamide;
4-((3-chloro-1,4-dioxo-1,4-dihydronaphthalen-2-ylamino)methyl)-N-(quinolin-3-yl)benzamide;
4-((3-chloro-1,4-dioxo-1,4-dihydronaphthalen-2-ylamino)methyl)-N-(quinolin-5-yl)benzamide;
4-((3-chloro-1,4-dioxo-1,4-dihydronaphthalen-2-ylamino)methyl)-N-(2-methylquinolin-4-yl)benzamide;
4-((3-chloro-1,4-dioxo-1,4-dihydronaphthalen-2-ylamino)methyl)-N-(1H-indol-5-yl)benzamide;
4-((3-chloro-1,4-dioxo-1,4-dihydronaphthalen-2-ylamino)methyl)-N-(2-methyl-1H-indol-5-yl)benzamide;
4-((3-chloro-1,4-dioxo-1,4-dihydronaphthalen-2-ylamino)methyl)-N-(1H-indol-7-yl)benzamide;
4-((3-chloro-1,4-dioxo-1,4-dihydronaphthalen-2-ylamino)methyl)-N-(1H-indol-4-yl)benzamide;
4-((3-chloro-1,4-dioxo-1,4-dihydronaphthalen-2-ylamino)methyl)-N-(4-(4-ethylpiperazin-1-yl)phenyl)benzamide;
4-((3-chloro-1,4-dioxo-1,4-dihydronaphthalen-2-ylamino)methyl)-N-(1H-indazol-6-yl)benzamide;
4-((3-chloro-1,4-dioxo-1,4-dihydronaphthalen-2-ylamino)methyl)-N-(1H-pyrrolo[2,3-b]pyridin-5-yl)benzamide;
4-((3-chloro-1,4-dioxo-1,4-dihydronaphthalen-2-ylamino)methyl)-N-(1H-pyrazolo[3,4-b]pyridin-5-yl)benzamide;
4-((3-chloro-1,4-dioxo-1,4-dihydronaphthalen-2-ylamino)methyl)-N-(7-methyl-7H-pyrrolo[2,3-d]pyrimidin-4-yl)benzamide;
4-((3-chloro-1,4-dioxo-1,4-dihydronaphthalen-2-ylamino)methyl)-N-(2,3-dihydro-1H-inden-4-yl)benzamide;
4-(((3-bromo-1,4-dioxo-1,4-dihydronaphthalen-2-yl)amino)methyl)-N-(pyridin-2-yl)benzamide;
4-(((3-chloro-1,4-dioxo-1,4-dihydronaphthalen-2-yl)amino)methyl)-N-(pyrimidin-4-yl)benzamide;
4-(((3-chloro-1,4-dioxo-1,4-dihydronaphthalen-2-yl)amino)methyl)-N-(pyrazin-2-yl)benzamide;
4-((3-chloro-1,4-dioxo-1,4-dihydronaphthalen-2-ylamino)methyl)-N-(pyridin-4-ylmethyl)benzamide;
4-((3-chloro-1,4-dioxo-1,4-dihydronaphthalen-2-ylamino)methyl)-N-(2-morpholinoethyl)benzamide;
N-(2-(1H-indol-3-yl)ethyl)-4-((3-chloro-1,4-dioxo-1,4-dihydronaphthalen-2-ylamino)methyl)benzamide;
4-(((3-chloro-1,4-dioxo-1,4-dihydronaphthalen-2-yl)amino)methyl)-N-(2-(dimethylamino)ethyl)benzamide;
4-(((3-chloro-1,4-dioxo-1,4-dihydronaphthalen-2-yl)amino)methyl)-N-(2-(pyrrolidin-1-yl)ethyl)benzamide;
4-(((3-chloro-1,4-dioxo-1,4-dihydronaphthalen-2-yl)amino)methyl)-N-(2-(diethylamino)ethyl)benzamide;
4-(((3-chloro-1,4-dioxo-1,4-dihydronaphthalen-2-yl)amino)methyl)-N-(2-(piperidin-1-yl)ethyl)benzamide; and
4-(((3-chloro-1,4-dioxo-1,4-dihydronaphthalen-2-yl)amino)methyl)-N-(2-(4-methylpiperazin-1-yl)ethyl)benzamide;
or a pharmaceutically acceptable salt, hydrate or isomer thereof.

5. The method of claim 1, wherein the compound is 4-(((3-chloro-1,4-dioxo-1,4-dihydronaphthalen-2-yl)amino)methyl)-N-(pyridin-4-yl)benzamide, having the following formula: 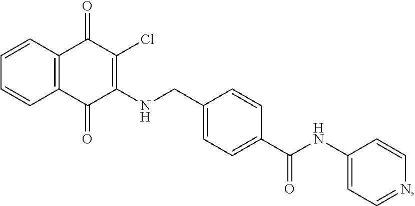 [SMILES: O=C(Nc1ccncc1)c1ccc(CNC2=C(Cl)C(=O)c3ccccc3C2=O)cc1]
or a pharmaceutically acceptable salt, hydrate or isomer thereof.

6. The method of claim 1, wherein the treatment and/or prevention of the optic neuropathy associated with apoptotic death of RGCs is via modulation of Nrf2 and TXNIP/NLRP3 inflammasome pathways in the retina.

7. The method of claim 1, wherein the compound enhances the expression of nuclear factor erythroid 2-related factor (Nrf2); downregulates thioredoxin interacting protein (TXNIP) expression, inhibits NLR family pyrin domain containing 3 (NLRP3) activation, and/or decreases inflammatory factors, interleukin (IL)-1P and IL-6 in the retina.

8. The method of claim 1, wherein the optic neuropathy associated with apoptotic death of RGCs is an optic nerve ischemic damage-related disease.

9. The method of claim 8, wherein the optic nerve ischemic damage-related disease is anterior ischemic optic neuropathy (AION) or posterior ischemic optic neuropathy (PION). In a further embodiment, the optic nerve ischemic damage-related disease is AION.

10. The method of claim 9, wherein the AION is nonarteritic ischemic optic neuropathy (NAION) or arteritic anterior ischemic optic neuropathy (AAION). In some further embodiments, the PION is nonarteritic posterior ischemic optic neuropathy (NPION) or arteritic posterior ischemic optic neuropathy (APION).

11. The method of claim 1, wherein the compound is administered subcutaneously.
